(19) United States
(12) Patent Application Publication
(10) Pub. No.: US 2012/0087990 A1
(43) Pub. Date: Apr. 12, 2012

(54) METHOD FOR PRODUCING ACTIVE HYDROGEN-DISSOLVED WATER AND APPARATUS FOR PRODUCING THE SAME

(75) Inventor: Seiki Shiga, Kanagawa (JP)

(73) Assignee: SHIGA FUNCTIONAL WATER LABORATORY CORPORATION, Sagamihara-shi, Kanagawa (JP)

(21) Appl. No.: 13/148,789

(22) PCT Filed: Feb. 4, 2010
(86) PCT No.: PCT/JP2010/000689

§ 371 Date: Nov. 17, 2011

(30) Foreign Application Priority Data
Feb. 10, 2009 (JP) 2009-028932
Feb. 4, 2010 (JP) 2010-023207

(51) Int. Cl. A61K 33/00; B01J 7/00; A61P 39/06

(52) U.S. Cl. 424/600; 422/129

(57) ABSTRACT

Provided are an apparatus and method for the long-term production of a drinking water in which active hydrogen is dissolved at a high concentration.

Specifically disclosed is a method for producing active hydrogen-dissolved water, which uses water of any one of the following items (1) to (3) containing at least any one of or both of calcium ion and magnesium ion; a hydrogen molecule dissociative adsorption catalyst that decomposes hydrogen molecules into active hydrogen in an aqueous solution; and a catalyst holding vessel that holds the hydrogen molecule dissociative adsorption catalyst, or uses water of any of the following items (1) to (3) containing at least any one of or both of calcium ion and magnesium ion; and a hydrogen molecule dissociative adsorption catalyst that decomposes hydrogen molecules into active hydrogen in an aqueous solution and retains the water for a certain time period, and which includes bringing the hydrogen molecule dissociative adsorption catalyst into contact with the water:
(1) water that has been in contact with magnesium metal;
(2) water in which hydrogen gas has been dissolved by bubbling or applying high pressure; and
(3) water that has been electrolyzed.

### FIELD

The present invention relates to a method and an apparatus for the production of active hydrogen-dissolved water.

### BACKGROUND

In recent years, active hydrogen-dissolved water containing a large amount of active hydrogen as compared with conventional drinking water, has attracted attention since the active hydrogen-dissolved water reduces active oxygen in the living body and is highly effective in the promotion of human health.

Thus, in order to supply the active hydrogen-dissolved water to consumers more conveniently at low cost, there is known a very convenient method for producing active hydrogen-dissolved water. This method involves allowing drinking water to react with magnesium metal (granules) as shown in Reaction Scheme 1 to generate active hydrogen, and thereby converting the drinking water to active hydrogen-dissolved water richly containing active hydrogen.

[Chemical formula 1]

Mg+2H₂O→Mg(OH)₂+2H→Mg(OH)₂+H₂ (1)

However, when active hydrogen-dissolved water is produced according to a method using the active hydrogen generating reaction based on magnesium metal, a magnesium hydroxide film is generated on the surface of magnesium metal, and therefore, it has been difficult to provide active hydrogen-dissolved water stably for a long time.

Thus, as a method of suppressing the generation of a magnesium hydroxide film on the surface of magnesium metal, the method of Patent Document 1 has been disclosed. In the method of Patent Document 1, the generation of a magnesium hydroxide film is suppressed by adding calcium sulfate, and thereby the active hydrogen generation capacity is maintained for a long time.

However, in the technologies of the related art, there is a problem that the action by which active hydrogen is converted back to hydrogen molecules may not be inhibited (apparent inhibition), and the hydrogen in the active hydrogen-dissolved water cannot be fully utilized.

### RELATED ART

### Patent Document

Patent Document 1: Japanese Patent Application Laid-Open (JP-A) No. 2006-255613

### DISCLOSURE OF INVENTION

### Problem to be Solved by Invention

It is an object of the present invention to address the problems described above, and to provide an apparatus and a method for the long-term production of a drinking water in which active hydrogen is dissolved at a high concentration.

### Means for Solving Problem

A method for producing active hydrogen-dissolved water of the present invention is characterized by bringing a hydrogen molecule dissociative adsorption catalyst into contact with a water;

the water being any one of the following items (1) to (3), containing at least any one of or both of calcium ion and magnesium ion;

the hydrogen molecule dissociative adsorption catalyst decomposing a hydrogen molecule into an active hydrogen in an aqueous solution; and

being provided with a catalyst holding vessel holding the hydrogen molecule dissociative adsorption catalyst,

or

the water being any of the following items (1) to (3), containing at least any one of or both of calcium ion and magnesium ion; and

the hydrogen molecule dissociative adsorption catalyst decomposing a hydrogen molecule into an active hydrogen in an aqueous solution and retaining the water for a certain time period,

(1) a water being in contact with magnesium metal;

(2) a water dissolving hydrogen gas, by bubbling or applying high pressure; and

(3) water being electrolyzed.

In the method for producing active hydrogen-dissolved water of the present invention, the water of the items (2) and (3) is a water that has been in contact with magnesium metal.

In the method for producing active hydrogen-dissolved water of the present invention, the water of the items (1) to (3) is further brought into contact with magnesium metal inside the catalyst holding vessel.

In the method for producing active hydrogen-dissolved water of the present invention, the hydrogen molecule dissociative adsorption catalyst contains one or more selected from the group consisting of palladium, platinum, rhodium, ruthenium, zinc, zirconium, titanium, hafnium, vanadium, niobium, tungsten, iron, ruthenium oxide, rhodium oxide, copper oxide, zinc oxide, zirconium oxide, silicon dioxide, titanium oxide, hafnium oxide, aluminum oxide, vanadium oxide, niobium oxide, tungsten oxide, and iron oxide.

In the method for producing active hydrogen-dissolved water of the present invention, the hydrogen molecule dissociative adsorption catalyst contains one or more metal oxides selected from the group consisting of silicon dioxide, aluminum oxide, zirconium oxide, and titanium dioxide.

In the method for producing active hydrogen-dissolved water of the present invention, the hydrogen molecule dissociative adsorption catalyst has been subjected to an acid treatment in advance.

In the method for producing active hydrogen-dissolved water of the present invention, the acid treatment is conducted with an acid at a pH in the range of from 2.5 or more to 4.5 or less.

The active hydrogen generator of the present invention is characterized by including a hydrogen molecule dissociative adsorption catalyst which decomposes hydrogen molecules into active hydrogen, and

a catalyst holding vessel that holds the hydrogen molecule dissociation adsorption catalyst, or

characterized by including a hydrogen molecule dissociative adsorption catalyst which decomposes hydrogen molecules into active hydrogen in an aqueous solution and retains the water for a certain time period.

In the active hydrogen generator of the present invention, magnesium metal is further included in the catalyst holding vessel that holds the hydrogen molecule dissociation adsorption catalyst.

In the active hydrogen generator of the present invention, the hydrogen molecule dissociative adsorption catalyst is one or more selected from the group consisting of palladium, platinum, rhodium, ruthenium, zinc, zirconium, titanium, hafnium, vanadium, niobium, tungsten, iron, ruthenium oxide, rhodium oxide, copper oxide, zinc oxide, zirconium oxide, silicon dioxide, titanium oxide, hafnium oxide, aluminum oxide, vanadium oxide, niobium oxide, tungsten oxide, and iron oxide.

In the active hydrogen generator of the present invention, one or more selected from the group consisting of calcium sulfate anhydride, calcium sulfate hemihydrate, and calcium sulfate dehydrate is further disposed in the active hydrogen generator.

In the active hydrogen generator of the present invention, the hydrogen molecule dissociative adsorption catalyst is a hydrogen molecule dissociative adsorption catalyst containing at least a solid acid.

In the active hydrogen generator of the present invention, the solid acid is one or more selected from the group consisting of silicon dioxide, aluminum oxide, zirconium oxide, and titanium dioxide.

### Effects of Invention

When a hydrogen molecule dissociative adsorption catalyst is used in the production of the active hydrogen-dissolved water, it becomes possible to produce water in which active hydrogen is dissolved at a high concentration for a longer time.

### BRIEF DESCRIPTION OF THE DRAWINGS

FIG. 1 is a conceptual diagram showing an example of the active hydrogen generator of the present invention;

FIG. 2 is a conceptual diagram showing an example of the active hydrogen generator of the present invention;

FIG. 3 is a conceptual diagram showing an example of the active hydrogen generator of the present invention; and

FIG. 4 is a graph showing the concentration of dissolved hydrogen of Example 1.

### BEST MODE(S) FOR CARRYING OUT THE INVENTION

The inventors of the present invention conducted extensive various investigations in order to obtain active hydrogen-dissolved water in which active hydrogen is dissolved at a higher concentration for a longer time, and thus the inventors found the following facts.

First, the inventors found that when active hydrogen-dissolved water is brought into contact with a metal or metal oxide having hydrogen molecule dissociative adsorption catalytic ability as a hydrogen molecule dissociative adsorption catalyst that decomposes hydrogen molecules into active hydrogen, the concentration of active hydrogen in the active hydrogen-dissolved water can be increased in the presence of a divalent alkaline earth metal ion.

Furthermore, even the active hydrogen obtained by the decomposition of hydrogen molecules goes back to the molecular state of hydrogen in a short time. Thus, the inventors found a method of making active hydrogen that has been generated by the decomposition of molecular hydrogen as a result of the contact with a hydrogen molecule dissociative adsorption catalyst, to be present in active hydrogen-dissolved water at a high concentration for a long time.

The active hydrogen-dissolved water of the present invention contains at least any one of or both of calcium ion and magnesium ion, and can be produced by bringing water or hydrogen-rich water that has been in contact with magnesium metal, into contact with a hydrogen molecule dissociative adsorption catalyst.

the water used in the present invention is preferably water such as tap water, electrically decomposed water, or mineral water, or a water obtained by adding calcium ions or magnesium ions to these kinds of water or to purified water. Use may also be made of a hydrogen-rich water in which hydrogen is dissolved at a high concentration, which has been prepared by mixing hydrogen gas (hydrogen molecules) with water by means of bubbling or the like, or by filling a water-containing vessel with hydrogen gas (hydrogen molecules) and applying high pressure. In the case of hydrogen-rich water, in order to maintain the dissolved concentration of hydrogen, a vessel which does not easily release hydrogen molecules to the atmosphere, such as an aluminum pouch, may be used as the vessel that holds the active hydrogen-dissolved water. When purified water such as ion-exchanged water is used, it is necessary to add water-soluble salts of calcium or magnesium to water in order to supplement calcium ions or magnesium ions.

Further for water with less mineral contents, such as soft water or ultrasoft water, it is preferable to add these salts to water for the purpose of increasing the dissolved concentration of active hydrogen.

For the water used in the present invention, if the water is not hydrogen-rich water, it is needed to convert the water into water in which active hydrogen is dissolved, by bringing the water into contact with magnesium metal. Furthermore, even hydrogen-rich water may be further brought into contact with magnesium metal for the purpose of increasing the concentrations of active hydrogen and hydrogen. Contacting of water with magnesium metal may be carried out either at the time of bringing water into contact with the hydrogen molecule dissociative adsorption catalyst, or before bringing water into contact with the hydrogen molecule dissociative adsorption catalyst.

Magnesium metal and water generate active hydrogen through the reaction shown below (Reaction Scheme 1). When active hydrogen is generated, magnesium metal is converted to magnesium hydroxide.

[Chemical Formula 2]

Mg+2H₂O→Mg(OH)₂+2H→Mg(OH)₂+H₂ (1)

Reaction Scheme 1

The ion of a divalent alkaline earth metal such as calcium ion or magnesium ion is a constituent element for stabilizing the generated active hydrogen by magnesium metal. According to the present invention, it is believed that active hydrogen is stabilized by a hydrated ion of a divalent alkaline earth metal ion. When active hydrogen is stabilized by a divalent alkaline earth metal ion, it is made possible to maintain a high concentration of active hydrogen in an aqueous solution for a period of 10 hours or longer. In order to be taken into the living body with high efficiency, the divalent alkaline earth metal ion is preferably any of or both of calcium ion and magnesium ion, which are dissolved in the living body (body fluid) at relatively high concentrations. Since these metal ions are internally taken into cells through the ion channels of the cellular membrane, it is speculated that stabilized active hydrogen is taken into the cells together with these metal ions. Monovalent sodium ion and potassium ion are also internally taken into the cells through the ion channels of the cellular membrane, but these ions are not capable of stabilizing active hydrogen. Therefore, calcium ions or magnesium ions are essential for the active hydrogen-dissolved water.

Stabilized active hydrogen undergoes an increase in the velocity of molecular movement under the action of energy such as heat. Therefore, it is not preferable to apply large energy to the aqueous solution by boiling the active hydrogen-dissolved water or the like, except for the occasion of maintenance of the active hydrogen generator.

When any of calcium sulfate and magnesium sulfate, or both of the salts are provided to the system of generating active hydrogen, a reaction represented by the following Reaction Scheme 2 or 3 proceeds, and the reaction suppresses the formation of magnesium hydroxide on the surface of magnesium metal.

[Chemical Formula 3]

Mg(OH))₂+Ca²⁺+SO₄²⁻→Mg²⁺+SO₄²⁻+Ca²⁺+2OH⁻ (2)

Reaction Scheme 2

[Chemical Formula 4]

Mg(OH)₂+Mg²⁺+SO₄²⁻→2Mg²⁺+SO₄²⁻+2OH⁻ (3)

Reaction Scheme 3

Calcium sulfate may be any of or two or more kinds of an anhydride, a hemihydrate and a hydrate, (hereinafter, hydrates and anhydride will be briefly described as calcium sulfate). In the case of including calcium sulfate into the active hydrogen generator, granular or rod-like calcium sulfate, or calcium sulfate in the form of an aggregate obtained by mixing calcium sulfate with magnesium metal or the like and press molding, may be used.

As the hydrogen molecule dissociative adsorption catalyst that decomposes dissolved hydrogen molecules into active hydrogen in water, use can be made of one or more kinds selected from the group of metals consisting of palladium, platinum, rhodium, ruthenium, zinc, zirconium, titanium, hafnium, vanadium, niobium, tungsten and iron, and oxides consisting of ruthenium oxide, rhodium oxide, copper oxide, zinc oxide, zirconium oxide, silicon dioxide, titanium oxide, hafnium oxide, aluminum oxide, vanadium oxide, niobium oxide, tungsten oxide and iron oxide, which are described in the Reference Literature (J. R. Anderson, Structure of Metallic Catalysis, p. 14, Academic Press (1975); and Catalyst Chemistry, edited by Keii, Tominaga, p. 131, Tokyo Kagaku Dojin, (1981)).

In addition, a metal having low adsorption energy of hydrogen molecules, such as copper, is less effective in increasing the amount of dissolved active hydrogen. Therefore, such a metal is not suitable as the active hydrogen molecule dissociative adsorption catalyst of the present invention.

Such a hydrogen molecule dissociative adsorption catalyst may be the catalyst substance alone, or may be a material having such a catalyst substance supported on another material. Furthermore, a mixture or the like of a ceramic or a mineral containing a catalyst substance having a hydrogen molecule dissociative adsorption catalytic action may also be used. As the material for supporting the catalyst substance, a structural material such as a ceramic material or a plastic material can be used. It is necessary that such a material be a material that is not soluble in water.

Among the hydrogen molecule dissociative adsorption catalysts listed above, a ceramic containing zirconium oxide and titanium oxide is more preferred. The catalytic capacity for hydrogenolysis of this catalyst for the use in active hydrogen generation is superior to the catalytic ability of platinum, and the catalyst is inexpensive, which is preferable.

It is preferable that the hydrogen molecule dissociative adsorption catalyst that decomposes hydrogen molecules into active hydrogen, be sparingly soluble in water. Furthermore, it is preferable to use a hydrogen molecule dissociative adsorption catalyst that is innocuous, or almost innocuous, to the human body even if a small amount is ingested.

In the case of using the hydrogen molecule dissociative adsorption catalyst in the state of being supported on a ceramic, the hydrogen molecule dissociative adsorption catalyst is preferably in the form in which the hydrogen molecule dissociative adsorption catalyst is supported on the ceramic by means of sintering a ceramic raw material containing the metal or metal oxide described above as the hydrogen molecule dissociative adsorption catalyst (hydrogen molecule dissociative adsorption catalyst substance), or a precursor thereof. There are no particular limitations on the content of the hydrogen molecule dissociative adsorption catalyst, but if the hydrogen molecule dissociative adsorption catalytic ability is too low, the effect of the hydrogen molecule dissociative adsorption catalyst is reduced. Thus, it is preferable that the hydrogen molecule dissociative adsorption catalyst be contained in an amount of 10 wt % or more based on the ceramic material supporting the hydrogen molecule dissociative adsorption catalyst.

The method of supporting a hydrogen molecule dissociative adsorption catalyst on a ceramic is preferably carried out by a method of mixing any one of a hydrogen molecule dissociative adsorption catalyst substance and a precursor thereof, or both of them, with the raw material of the ceramic, and sintering the mixture; or a method of applying the hydrogen molecule dissociative adsorption catalyst substance on the ceramic by means of sand blasting, plating or the like. In the case of performing an acid treatment that will be described below, the area for supporting the hydrogen molecule dissociative adsorption catalyst on the ceramic is preferably from 20% to 80% of the surface area of the ceramic material. If the area is larger than 80%, the economic efficiency is not better, and if the area is smaller than 20%, the hydrogen molecule dissociative adsorption catalytic ability is lowered, which is not preferable.

In order to further increase the concentration of active hydrogen in the active hydrogen-dissolved water, it is necessary to cause hydrated ions, which stabilize active hydrogen that has been generated by the hydrogen molecule dissociative adsorption catalyst, to be present in the vicinity of the catalyst. When a solid acid is used as the hydrogen molecule dissociative adsorption catalyst, and the surface of the hydrogen molecule dissociative adsorption catalyst is electrically negatively charged in an aqueous solution in the neutral region, calcium ions or magnesium ions coordinating water molecules are caused to interact with the catalyst by electrostatic attraction, and thereby the probability of bringing these ions to be present in the vicinity of the catalyst is increased. It is speculated that when the catalyst and the calcium ions or magnesium ions are present in the neighborhood, the active hydrogen generated by the hydrogen molecule dissociative adsorption catalyst can be more easily stabilized by the hydrated ions of calcium or magnesium.

Such hydrogen molecule dissociative adsorption catalyst with negatively charged surface is more likely to bring in the hydrated ions of an alkaline earth metal (positive) such as calcium or magnesium to the neighborhood through electrical attraction, and thus the probability that the hydrated ions are present in the vicinity of the hydrogen molecule dissociative adsorption catalyst is increased. Furthermore, since the hydrated ions that are believed to adsorb active hydrogen are likely to be present in the vicinity of the hydrogen molecule dissociative adsorption catalyst, the probability that the hydrated ions adsorb the active hydrogen that has been generated by the hydrogen molecule dissociative adsorption catalyst is also increased.

The raw materials of the hydrogen molecule dissociative adsorption catalyst may include solid bases. When a solid base is included in the hydrogen molecule dissociative adsorption catalyst, the highest acid strength or the degree of acidity possessed by the hydrogen molecule dissociative adsorption catalyst is decreased. Therefore, there is a risk that the hydrogen molecule dissociative adsorption catalytic activity or the negativity of the charge carried by the hydrogen molecule dissociative adsorption catalyst may be decreased. Thus, it is preferable to subject the hydrogen molecule dissociative adsorption catalyst to an acid treatment and to thereby eliminate the solid base from the hydrogen molecule dissociative adsorption catalyst.

Examples of the solid base that can be easily removed by subjecting the hydrogen molecule dissociative adsorption catalyst to an acid treatment include calcium oxide, magnesium oxide, potassium oxide, sodium oxide, and the like.

When a solid base is included in the hydrogen molecule dissociative adsorption catalyst, it is preferable to perform the acid treatment as described above, and it is preferable to dissolve the solid base with an acid which would not dissolve a solid acid. Therefore, when a solid base is included in the raw materials on the occasion of producing a hydrogen molecule dissociative adsorption catalyst, it is preferable to use raw materials in which the solid base is acid-soluble, and more preferably highly acid-soluble.

Furthermore, when copper, copper oxide or the like is included in the hydrogen molecule dissociative adsorption catalyst, it is speculated that when these substances are dissolved, the hydrogen molecule dissociative adsorption catalyst becomes more acidic. Therefore, it is also preferable to dissolve copper, copper oxide and the like by an acid treatment, in the same manner as for the case of solid base.

The method for the acid treatment is preferably carried out by any one of or by both of a method of immersing the hydrogen molecule dissociative adsorption catalyst in an acid solution that has been adjusted to a predetermined pH value, and a method of washing the hydrogen molecule dissociative adsorption catalyst with an acid solution. The acid treatment is dependent on the pH of the acid solution; however, in order to sufficiently perform the acid treatment, in the case of immersing the hydrogen molecule dissociative adsorption catalyst in an acid solution, it is preferable to immerse the catalyst for 7 minutes or longer, and in the case of washing the hydrogen molecule dissociative adsorption catalyst with an acid solution, it is preferable to wash the catalyst several times. Furthermore, in the case of performing the acid treatment through immersion, the acid treatment may be carried out while the system is stirred using a stirrer or the like. After the acid treatment has been performed, it is preferable to sufficiently rinse the catalyst with tap water or the like.

The acid used in the acid treatment is preferably an acid that dissolves the solid base present in the hydrogen molecule dissociative adsorption catalyst, does not dissolve a solid acid, and has no adverse effect on the activity of the hydrogen molecule dissociative adsorption catalyst. In addition, since it is still acceptable to have a pH that is not equal to or lower than the acidic center of the solid acid, the acid used in the acid treatment is preferably an acid having a pH value of from 2.5 or more to 4.5 or less. Among others, an acid having a pH value of about 3.5 is more preferable. As the acid having a pH value of from 2.5 or more to 4.5 or less, it is preferable to use a solution which has been prepared using one or more kinds of sulfuric acid, nitric acid, hydrochloric acid, phosphoric acid, acetic acid, oxalic acid, lactic acid, formic acid, citric acid and the like in accordance with the solid acid or solid base included in the hydrogen molecule dissociative adsorption catalyst, and adjusting them to a predetermined pH, in the acid treatment.

Examples of the solid acid used in the present invention include known solid acids. Among them, when acid resistance and the hydrogen molecule dissociative adsorption catalytic ability are taken into consideration, the solid acid is preferably a metal oxide such as silicon dioxide, aluminum oxide, zirconium oxide, or titanium dioxide.

It is desirable to mix raw materials including the simple substance of the solid acid that is contained in the hydrogen molecule dissociative adsorption catalyst, or a precursor of the solid acid, form the mixture into an arbitrary shape, and fire the formed mixture.

Next, some examples of the configuration of the active hydrogen generator will be described. The fundamental configuration of the active hydrogen generator includes a hydrogen molecule dissociative adsorption catalyst which decomposes hydrogen molecules into active hydrogen, and a catalyst holding vessel which holds the hydrogen molecule dissociative adsorption catalyst. Examples of the catalyst holding vessel include drinking vessels such as PET bottles, vessels that are introduced into drinking vessels, and vessel capable of retaining the hydrogen molecule dissociative adsorption catalyst at a certain place, such as a tank for collecting water. In addition to that, there is another form of the active hydrogen generator having, as the fundamental configuration, a hydrogen molecule dissociative adsorption catalyst which also functions as a catalyst holding vessel such as one used in the filter layer of a filtering device such as a water purifier, in which the hydrogen molecule dissociative adsorption catalyst is disposed in a region (space) that is separated by a filter or the like in an aqueduct such that the hydrogen molecule dissociative adsorption catalyst retains water for a certain time period and can be in contact with the retained water.

Particularly, when the catalyst holding vessel is a vessel which has a risk that the hydrogen molecule dissociative adsorption catalyst may be accidentally swallowed, such as a drinking vessel or a vessel that is introduced into a drinking vessel, it is preferable to provide a filter in the vicinity of the drinking faucet in order to prevent accidental swallowing. It is necessary for the active hydrogen generator of the present invention to retain water for a certain time period, so that the hydrogen molecule dissociative adsorption catalyst decomposes hydrogen molecules into hydrogen atoms. This certain time period may vary with the form of the active hydrogen generator, but it is desirable that the time period be a period of time during which water containing calcium ions or magnesium ions and also containing active hydrogen or hydrogen molecules can be in contact with the hydrogen molecule dissociative adsorption catalyst. Therefore, a vessel that retains water only for a very short time, such as the filter of a water purifier, may also be used. It is preferable that this certain time period be longer because the time period is a period of time during which hydrogen molecules are decomposed into hydrogen atoms. Furthermore, in the vessel for holding the hydrogen molecule dissociative adsorption catalyst, a translucent member for visualization may also be used at least in a part so that the dissolved state of magnesium metal, or the like can be easily visually observed from the outside.

Examples of the form of the hydrogen molecule dissociative adsorption catalyst include, in the case of using a catalyst holding vessel, forms in which the hydrogen molecule dissociative adsorption catalyst is made to be present inside the system for generating active hydrogen, such as a form in which particulate objects and the like are placed in the inside of the catalyst holding vessel, a form in which the catalyst is attached to the external side of the catalyst holding vessel, and a form in which at least a part of the catalyst holding vessel is constructed with the hydrogen molecule dissociative adsorption catalyst.

In the case where the hydrogen molecule dissociative adsorption catalyst itself also functions as the catalyst holding vessel, examples of the form of the hydrogen molecule dissociative adsorption catalyst include a form in which the catalyst is used in an aqueduct through which water flows, a form in which a catalyst region is formed in a part of the aqueduct, and a form in which the catalyst is used for the vessel itself for collecting water.

In the following, the form of the active hydrogen generator will be described by way of the attached drawings.

The size and shape of the materials in the conceptual diagrams that will be described below are only exemplary, and the size and shape are not intended to be limited to those shown in the drawings.

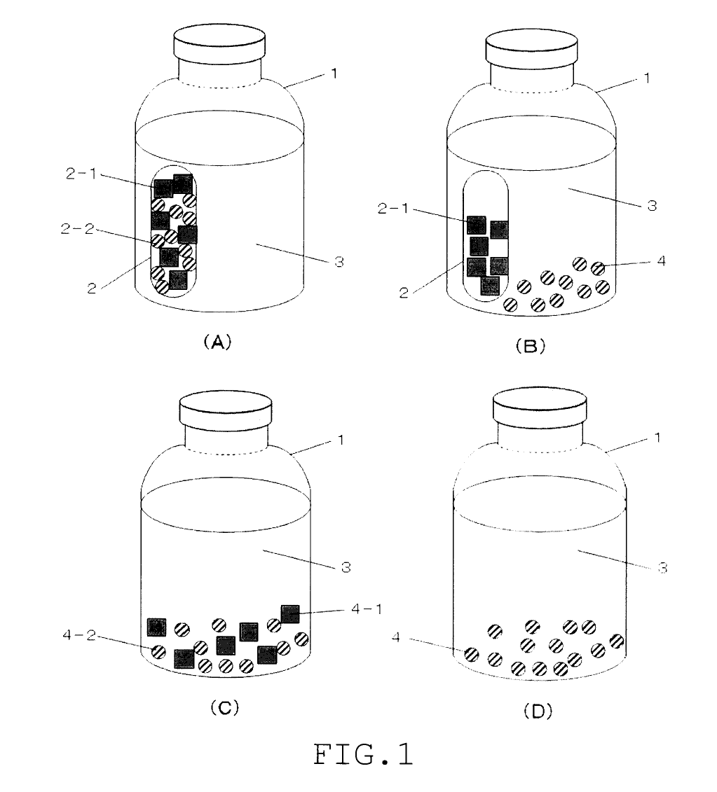

For example, a first active hydrogen generator will be described. The concept of the first form of active hydrogen generator is a form in which the hydrogen molecule dissociative adsorption catalyst or magnesium metal is placed in a vessel holding water. A specific example is a form in which magnesium metal 2-1 and the hydrogen molecule dissociative adsorption catalyst 2-2 have been inserted into an active hydrogen generating vessel (catalyst holding vessel) 2 having numerous fine pores, which is disposed in a drinking vessel 1 containing water 3, and FIG. 1(A) presents a conceptual diagram thereof. The drinking vessel 1 is, for example, a vessel made of plastic. The drinking vessel is constructed such that water 3, active hydrogen or the like can move through the fine pores of the active hydrogen generating vessel 2.

Another specific example may be a form in which, unlike FIG. 1(A), the hydrogen molecule dissociative adsorption catalyst 4 is provided on the external side of the active hydrogen generating vessel 2, and the drinking vessel 1 is used as the catalyst holding vessel according to the present invention, as shown in the conceptual diagram of FIG. 1(B). Furthermore, calcium sulfate may also be added to the active hydrogen generating vessel 2.

Another specific example may be a form in which, unlike FIG. 1(A), a vessel for holding the hydrogen molecule dissociative adsorption catalyst only is not used, but the hydrogen molecule dissociative adsorption catalyst is disposed in the drinking vessel (catalyst holding vessel) 1 such as a PET bottle by directly adding magnesium metal 4-1 and the hydrogen molecule dissociative adsorption catalyst 4-2 to water 3, as shown in the conceptual diagram of FIG. 1(C).

Another specific example may be a form in which, unlike FIG. 1(A), a vessel for holding the hydrogen molecule dissociative adsorption catalyst only is not used, but the hydrogen molecule dissociative adsorption catalyst 4 is disposed in the drinking vessel (catalyst holding vessel) 1 such as a PET bottle by directly adding the hydrogen molecule dissociative adsorption catalyst 4 to water 3, as shown in the conceptual diagram of FIG. 1(D).

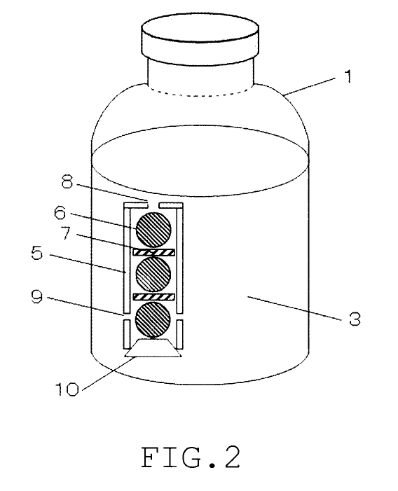

For example, a second active hydrogen generator will be described. The concept of the second form of active hydrogen generator is a modification example of the active hydrogen generating vessel used in the first form of active hydrogen generator. It is a form in which aggregates 6 of magnesium metal and calcium sulfate, and the hydrogen molecule dissociative adsorption catalyst 7 have been inserted into an active hydrogen generating vessel (catalyst holding vessel) 5 having openings 8 and 9 at the top and bottom and a stopper 10, which is disposed in a drinking vessel 1 containing water 3, and FIG. 2 presents a conceptual diagram of the relevant active hydrogen generating vessel. When the movement of water and active hydrogen is taken into consideration, it is preferable that there be one or more of the opening 8 provided at the top of the active hydrogen generating vessel, and two or more of the opening 9 provided at the bottom.

When the active hydrogen generating vessel 5 equipped with a stopper 10 as shown in FIG. 2, is used, it is easy to check the presence of remnants in the active hydrogen generating vessel or to supplement magnesium metal and calcium sulfate by removing the stopper 10.

In the case of providing the hydrogen molecule dissociative adsorption catalyst in other than the active hydrogen generating vessel, it is preferable for the hydrogen molecule dissociative adsorption catalyst to have a size or weight to the extent that the catalyst can be inserted through the drinking faucet of the catalyst holding vessel such as a PET bottle but cannot easily come out through the drinking faucet. For the purpose of enhancing the catalytic function, it is preferable that the hydrogen molecule dissociative adsorption catalyst has surface unevenness to a large extent. Furthermore, if the active hydrogen generating vessel floats in water, the active hydrogen generating vessel may be treated to prevent floating in the active hydrogen-dissolved water by attaching a weight to the active hydrogen generating vessel. A form in which magnesium metal or the hydrogen molecule dissociative adsorption catalyst is placed in another vessel which has holes and is likely to sink down, may also be used.

The active hydrogen generating vessel may be a vessel formed from a polymer such as porous sintered polyethylene, a metal such as stainless steel, or a material obtained by making the polymer or metal lightproof. Further, a vessel produced by using the hydrogen molecule dissociative adsorption catalyst that decomposes hydrogen molecules into active hydrogen, in a part or the entirety of the material for the active hydrogen generating vessel, may also be used, and in this case, the active hydrogen generating vessel itself can be utilized as the hydrogen molecule dissociative adsorption catalyst. Furthermore, in the case of using the hydrogen molecule dissociative adsorption catalyst in the active hydrogen generating vessel, it is preferable to make the active hydrogen generating vessel not easily destructible by taking a measure such as increasing the thickness of the active hydrogen generating vessel. It is preferable that the active hydrogen generating vessel have one or more holes, and that the size of the holes be a size through which at least active hydrogen and water can move in and out. Further, the size of the holes is preferably a size through which magnesium metal, the hydrogen molecule dissociative adsorption catalyst, and magnesium hydroxide cannot move out of the active hydrogen generating vessel. For this reason, although the size of the holes may depend on the thickness of the active hydrogen generating vessel, in the case of providing numerous fine holes, the size of the holes is preferably from 50 μm or more to 200 μm or less, and more preferably from 100 μm or more to 170 μm or less. In addition, in the case of providing aggregates of calcium sulfate and magnesium metal in the active hydrogen generating vessel, holes smaller than the aggregates are acceptable, and for example, the holes may be a few millimeters in size.

The size of the magnesium metal is preferably from 0.1 mm or more to 2.0 mm or less. If the size is smaller than 0.1 mm, magnesium metal may all be dissolve away in a short time, which is not preferable, and if the size is larger than 2.0 mm, there is a risk that magnesium metal may not completely dissolve, which is also not preferable.

The content ratio of the magnesium metal and the hydrogen molecule dissociative adsorption catalyst contained in the active hydrogen generator is preferably such that when the content of the magnesium metal is taken as 100 parts by mass, the content of the hydrogen molecule dissociative adsorption catalyst is 50 parts by mass or more. If the content ratio of the magnesium metal is low, there is a risk that the generation of active hydrogen may not occur sufficiently, which is not preferable.

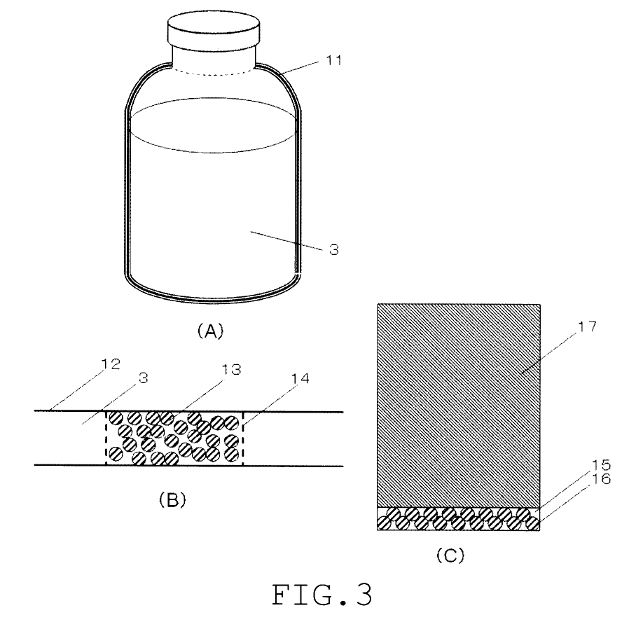

For example, a third form of the active hydrogen generator will be described. Unlike the first form and the second form, the concept of the third form of active hydrogen generator is a form which uses a hydrogen molecule dissociative adsorption catalyst capable of retaining water for a certain time in a vessel, a tube, a member or the like. Further, in the case of the third form of active hydrogen generator, magnesium metal may be used. The conceptual diagram of FIG. 3(A) shows a form in which the vessel containing water 3 is made into a hydrogen molecule dissociative adsorption catalyst vessel 11 formed from the hydrogen molecule dissociative adsorption catalyst. Magnesium metal, an active hydrogen generating vessel, or calcium sulfate, such as shown in the aforementioned forms of active hydrogen generator may be further added to the hydrogen molecule dissociative adsorption catalyst vessel 11.

Furthermore, as shown in the conceptual diagram of FIG. 3(B), a form in which the hydrogen molecule dissociative adsorption catalyst 13 is disposed inside an aqueduct 12 through which water 3 passes, may be used. In this form, a filter 14 is provided in order to maintain the hydrogen molecule dissociative adsorption catalyst 13 at the defined place. In this form, the region partitioned by the filter 14 constitutes the hydrogen molecule dissociative adsorption catalyst retaining water for a certain time period.

Further, a form in which the hydrogen molecule dissociative adsorption catalyst 16 is used in a part of the filter layer of a water purifier or the like, as shown in the conceptual diagram of FIG. 3(C), may be used, and in this example shown in the diagram, there is provided a hydrogen molecule dissociative adsorption catalyst layer 15 in which the hydrogen molecule dissociative adsorption catalyst 16 is disposed in the lower part of another filter layer 17. In this form, the hydrogen molecule dissociative adsorption catalyst layer 15 constitutes the hydrogen molecule dissociative adsorption catalyst retaining water for a certain time period.

### EXAMPLES

Hereinafter, the invention will be described in more detail by way of Examples.

Here, the dissolved hydrogen was measured using a dissolved hydrogen meter, KM2100DH type, manufactured by

Kyoei Denshi, Ltd. The dissolved hydrogen meter measures the total amount (mg/L) of the hydrogen molecule (H₂) or active hydrogen (H) described above.

### Examples 1 to 8 and Comparative Example 1

In the present exemplary embodiment, as shown in the conceptual diagram of FIG. 1(B), use is made of an active hydrogen generator which is equipped with an active hydrogen generating vessel 2 made of sintered polyethylene and charged with 7 g of magnesium metal 2-1 having an average particle size of 1.0 mm, and a catalyst holding vessel charged with 7 g of the hydrogen molecule dissociated adsorption catalyst particles 4 indicated in Table 1, and which is placed in a drinking vessel 1 such as a PET bottle. The active hydrogen generating vessel 2 of Example 1-3 made of sintered polyethylene has numerous pores having an average size of about 120 μm.

TABLE 1

| | Catalyst | Acid treatment |
|---|---|---|
| Example 1 | ZrO₂-containing ceramic | Treated |
| Example 2 | Platinum-supported ceramic | Untreated |
| Example 3 | CrO₂ ceramic | Untreated |
| Example 4 | CrO₂ ceramic | Treated |
| Example 5 | ZrO₂TiO₂-containing ceramic | Untreated |
| Example 6 | ZrO₂TiO₂-containing ceramic | Treated |
| Example 7 | W₂O ceramic | Untreated |
| Example 8 | W₂O ceramic | Treated |
| Comparative | Copper | Untreated |
| Example 1 | | |

The ZrO₂-containing ceramic used in the hydrogen molecule dissociative adsorption catalyst of the active hydrogen generator of Example 1 is a ceramic material having an average particle diameter of 4 mm, which was produced by mixing raw materials containing SiO₂, Al₂O₃ and Fe₂O₃ in a total amount of 60 wt % or more, containing CaO and MgO in a total amount of 5 wt % or less, and containing ZrO₂ in an amount of 30 wt %, and sintering the mixture. The ceramic material was washed several times with dilute hydrochloric acid at about pH 3.5, and the hydrogen molecule dissociative adsorption catalyst was thoroughly washed with tap water.

The platinum-supported ceramic used in the hydrogen molecule dissociative adsorption catalyst of the active hydrogen generator of Example 2 is a ceramic material having an average particle diameter of 4 mm, which was produced by plating a ceramic material of 99% alumina (99% Al₂O₃, 1% SiO₂, MgO, and Na₂O) with platinum having an average particle diameter of 10 μm such that the platinum would cover at least a portion of the ceramic surfaces.

The hydrogen molecule dissociative adsorption catalyst of Example 2 was not subjected to an acid treatment.

The CrO₂-containing ceramic used in the hydrogen molecule dissociative adsorption catalyst of the active hydrogen generator of Example 3 is a ceramic material having an average particle diameter of 4 mm, which was produced by mixing raw materials containing SiO₂, Al₂O₃ and

Fe₂O₃ in a total amount of 60 wt % or more, containing CaO and MgO in a total amount of 5 wt % or less, and containing CrO₂ in an amount of 30 wt %, and sintering the mixture.

The CrO₂-containing ceramic used in the hydrogen molecule dissociative adsorption catalyst of the active hydrogen generator of Example 4 is a ceramic material obtained by washing the CrO₂-containing ceramic of Example 4 several times with dilute hydrochloric acid at about pH 3.5, and thoroughly washing the hydrogen molecule dissociative adsorption catalyst with tap water.

The ZrO₂TiO₂-containing ceramic used in the hydrogen molecule dissociative adsorption catalyst of the active hydrogen generator of Example 5 is a ceramic material having an average particle diameter of 4 mm, which was produced by mixing raw materials containing SiO₂, Al₂O₃ and Fe₂O₃ in a total amount of 50 wt % or more, containing CaO and MgO in a total amount of 5 wt % or less, and containing ZrO₂ and TiO₂ in a total amount of 40 wt %, and sintering the mixture.

The ZrO₂TiO₂-containing ceramic used in the hydrogen molecule dissociative adsorption catalyst of the active hydrogen generator of Example 6 is a ceramic material obtained by washing the ZrO₂TiO₂-containing ceramic of Example 6 several times with dilute hydrochloric acid at about pH 3.5, and thoroughly washing the hydrogen molecule dissociative adsorption catalyst with tap water.

The W₂O-containing ceramic used in the hydrogen molecule dissociative adsorption catalyst of the active hydrogen generator of Example 7 is a ceramic material having an average particle diameter of 4 mm, which was produced by mixing raw materials containing SiO₂, Al₂O₃ and Fe₂O₃ in a total amount of 80 wt % or more, containing CaO and MgO in a total amount of 5 wt % or less, and containing W₂O in a total amount of 10 wt %, and sintering the mixture.

The W₂O-containing ceramic used in the hydrogen molecule dissociative adsorption catalyst of the active hydrogen generator of Example 8 is a ceramic material obtained by washing the W₂O-containing ceramic of Example 8 several times with dilute hydrochloric acid at about pH 3.5, and thoroughly washing the hydrogen molecule dissociative adsorption catalyst with tap water.

The copper used in the hydrogen molecule dissociative adsorption catalyst of the active hydrogen generator of Comparative Example 1 is a plate-shaped material having a size of about 100 mm×170 mm×0.1 mm.

The hydrogen molecule dissociative adsorption catalyst of Comparative Example 1 was not subjected to an acid treatment.

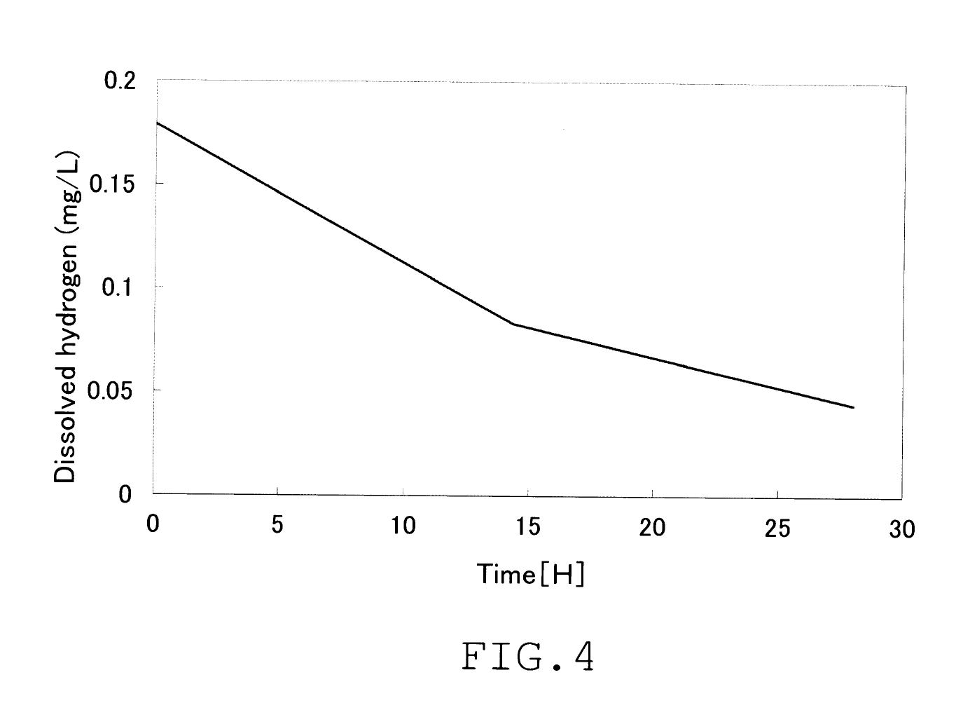

An active hydrogen generating vessel 2 containing 7 g of magnesium particles and 7 g of hydrogen molecule dissociative adsorption catalyst particles (about 16 g for Comparative Example 1 only) was placed in a PET bottle containing 2000 mL of tap water, and the PET bottle was left to stand for 48 hours at room temperature. Subsequently, the active hydrogen generator was removed from the PET bottle, and changes in the amount of dissolved hydrogen [mg/L] (the sum of hydrogen molecules and active hydrogen) over time were examined. The results are presented in FIG. 4.

From the graph of FIG. 4 showing the changes over time in the amount of dissolved hydrogen of Example 1, it can be seen that there is an inflection point about 15 hours after the removal of the active hydrogen generator. Before this inflection point, a decrease in the amount of active hydrogen and hydrogen molecules is shown, and after this inflection point, a decrease in the amount of hydrogen molecules is exhibited. The vertical axis of the graph in FIG. 4 is expressed in a logarithmic scale. Therefore, the value (Bx) of the segment of the fitted curve obtained from the plot data of the amount of dissolved hydrogen after the inflection point represents the amount of dissolved hydrogen molecules (Bx) at the time when the active hydrogen generator was removed from the PET bottle, and the value obtained by subtracting the amount of dissolved hydrogen molecules (Bx) from the amount of dissolved hydrogen (Ax) at the time when the active hydrogen generator was removed from the PET bottle, represents the amount of dissolved active hydrogen (Cx) at the time when the active hydrogen-dissolved water generating vessel and the hydrogen molecule dissociative adsorption catalyst were removed from the PET bottle.

The amounts of dissolved hydrogen (Ax), the amounts of dissolved hydrogen molecules (Bx), and the amounts of dissolved active hydrogen (Cx) of Examples 1 to 8 and Comparative Example 1 at the time when the active hydrogen generator was removed from the PET bottle are presented in Table 2.

TABLE 2

| | Ax | Bx | Cx | Cx/Ax [%] |
|---|---|---|---|---|
| Example 1 | 0.180 | 0.130 | 0.050 | 28% |
| Example 2 | 0.110 | 0.080 | 0.030 | 27% |
| Example 3 | 0.350 | 0.260 | 0.090 | 26% |
| Example 4 | 0.350 | 0.220 | 0.130 | 37% |
| Example 5 | 0.350 | 0.230 | 0.120 | 34% |
| Example 6 | 0.350 | 0.200 | 0.150 | 43% |
| Example 7 | 0.300 | 0.190 | 0.110 | 37% |
| Example 8 | 0.320 | 0.220 | 0.100 | 31% |
| Comparative | 0.180 | 0.135 | 0.045 | 25% |
| Example 1 | | | | |

### Examples 9 to 12 and Comparative Example 2

According to the present exemplary embodiment, as shown in the conceptual diagram of FIG. 2, use is made of an active hydrogen generator which is equipped with an active hydrogen generating vessel 5 made of plastic and charged with magnesium metal, calcium sulfate and a hydrogen molecule dissociative adsorption catalyst at a mass ratio of 10:1:10 (15 g in total), and which is placed in a drinking vessel 1 such as a PET bottle. The active hydrogen generating vessel 5 is charged with aggregates 6 in which magnesium metal having an average particle diameter of 0.2 mm is included in calcium sulfate having an average particle diameter of 10 mm, and hydrogen molecule dissociative adsorption catalyst particles 7. The active hydrogen generating vessel 5 made of plastic has openings each having a size of 1.5 mm for water conduction, one opening at the top and two to four openings at the bottom.

TABLE 3

| | Catalyst | Acid treatment |
|---|---|---|
| Example 9 | ZrO₂-containing ceramic | Treated |
| Example 10 | Platinum-supported ceramic | Untreated |
| Example 11 | Titanium-supported ceramic | Untreated |
| Example 12 | Brass | Untreated |
| Comparative | No catalyst | Untreated |
| Example 2 | | |

The ZrO₂-containing ceramic used in the hydrogen molecule dissociative adsorption catalyst of the active hydrogen generator of Example 9 is the same hydrogen molecule dissociative adsorption catalyst as that used in Example 1.

The platinum-supported ceramic used in the hydrogen molecule dissociative adsorption catalyst of the active hydrogen generator of Example 10 is the same hydrogen molecule dissociative adsorption catalyst as that used in Example 2.

The titanium-supported ceramic used in the hydrogen molecule dissociative adsorption catalyst of the active hydrogen generator of Example 11 is a ceramic having an average particle diameter of 5 mm, which was produced by spraying titanium having an average particle diameter of 50 μm on a ceramic of 99% alumina with a sand blast, such that the titanium covered about 80% of the ceramic surfaces.

The hydrogen molecule dissociative adsorption catalyst of Example 11 was not subjected to an acid treatment.

The brass used in the hydrogen molecule dissociative adsorption catalyst of the active hydrogen generator of Example 12 is a granular material having an average particle diameter of 5 mm.

The hydrogen molecule dissociative adsorption catalyst of Example 12 was not subjected to an acid treatment.

In the Comparative Example 2, an active hydrogen generator which was not equipped with a metal or metal oxide that would serve as the hydrogen molecule dissociative adsorption catalyst of the active hydrogen generator, was used.

Two pieces each of the active hydrogen generating vessels of Examples 9 to 12 and Comparative Example 2 were placed in a PET bottle containing 2000 mL of tap water, and the PET bottle was left to stand for 48 hours at room temperature. Subsequently, the amounts of dissolved hydrogen were analyzed in the same manner as in Example 1, and thus Ax, Bx and Cx were determined. A summary of the values of Ax, Bx and Cx is presented in Table 4.

TABLE 4

| | Ax | Bx | Cx | Cx/Ax [%] |
|---|---|---|---|---|
| Example 9 | 0.085 | 0.050 | 0.035 | 41% |
| Example 10 | 0.070 | 0.040 | 0.030 | 43% |
| Example 11 | 0.085 | 0.055 | 0.030 | 35% |
| Example 12 | 0.070 | 0.045 | 0.025 | 36% |
| Comparative | 0.085 | 0.060 | 0.025 | 29% |
| Example 2 | | | | |

### Examples 13 to 20 and Comparative Examples 3 to 4

In the present exemplary embodiment, as shown in the conceptual diagram of FIG. 1(A), use is made of an active hydrogen generator which is equipped with an active hydrogen generating vessel 2 made of sintered polyethylene which is charged with magnesium metal 2-1 having an average particle diameter of 1.0 mm and the hydrogen molecule dissociative adsorption catalyst 2-2 of Table 5 at a mass ratio of 1:1, and which is placed in a drinking vessel 1 such as, for example, a PET bottle. The active hydrogen generating vessels 2 made of sintered polyethylene of Examples 13 to 20 and Comparative Examples 3 to 4 have numerous pores having an average size of about 120 μm.

TABLE 5

| | Catalyst | Acid treatment |
|---|---|---|
| Example 13 | ZrO₂-containing ceramic | Untreated |
| Example 14 | ZrO₂-containing ceramic | Treated |
| Example 15 | Platinum-supported ceramic | Treated |
| Example 16 | Titanium-supported alumina | Treated |
| | (60%) | |
| Example 17 | Titanium-supported alumina | Untreated |
| | (99%) | |
| Example 18 | Titanium-supported alumina | Treated |
| | (99%) | |
| Example 19 | Brass | Untreated |
| Example 20 | Tungsten | Untreated |
| Comparative | Copper | Untreated |
| Example 3 | | |
| Comparative | No catalyst | Untreated |
| Example 4 | | |

The ZrO₂-containing ceramic used in the hydrogen molecule dissociative adsorption catalyst of the active hydrogen generator of Example 13 is the same hydrogen molecule dissociative adsorption catalyst as that used in Example 1.

The ZrO₂-containing ceramic used in the hydrogen molecule dissociative adsorption catalyst of the active hydrogen generator of Example 14 is the same hydrogen molecule dissociative adsorption catalyst as that used in Example 1.

The platinum-supported ceramic used in the hydrogen molecule dissociative adsorption catalyst of the active hydrogen generator of Example 15 is the hydrogen molecule dissociative adsorption catalyst of Example 2 which has been subjected to an acid treatment in the same manner as in Example 1.

The titanium-supported ceramic used in the hydrogen molecule dissociative adsorption catalyst of the active hydrogen generator of Example 16 is a catalyst produced by supporting titanium on a ceramic of 60% alumina (60% Al₂O₃, 40% SiO₂, Fe₂O₃, CaO, MgO, K₂O, and Na₂O), and performing an acid treatment in the same manner as in Example 1.

The titanium-supported ceramic used in the hydrogen molecule dissociative adsorption catalyst of the active hydrogen generator of Example 17 is the same hydrogen molecule dissociative adsorption catalyst as that used in Example 11.

The titanium-supported ceramic used in the hydrogen molecule dissociative adsorption catalyst of the active hydrogen generator of Example 18 is a catalyst obtained by subjecting the hydrogen molecule dissociative adsorption catalyst of Example 11 to an acid treatment by the method of Example 1.

The brass used in the hydrogen molecule dissociative adsorption catalyst of the active hydrogen generator of Example 19 is the same as the hydrogen molecule dissociative adsorption catalyst of Example 12.

The tungsten used in the hydrogen molecule dissociative adsorption catalyst of the active hydrogen generator of Example 20 is a granular material having an average particle diameter of about 1.5 mm.

The copper used in the hydrogen molecule dissociative adsorption catalyst of the active hydrogen generator of Comparative Example 3 is a granular material having an average particle diameter of about 2 mm.

In Comparative Example 4, an active hydrogen generator which was not equipped with a metal or metal oxide that would serve as the hydrogen molecule dissociative adsorption catalyst of the active hydrogen generator, was used.

Two pieces each of the respective active hydrogen generating vessels of Examples 15 to 23 and Comparative Examples 3 and 4 were placed in a PET bottle containing 2000 mL of tap water, and the PET bottle was left to stand for 48 hours at room temperature. Subsequently, the amounts of dissolved hydrogen were analyzed in the same manner as in Example 1, and thus Ax, Bx and Cx were determined. A summary of the values of Ax, Bx and Cx is presented in Table 6.

TABLE 6

| | Ax | Bx | Cx | Cx/Ax [%] |
|---|---|---|---|---|
| Example 13 | 0.140 | 0.100 | 0.040 | 29% |
| Example 14 | 0.180 | 0.110 | 0.070 | 39% |
| Example 15 | 0.210 | 0.135 | 0.075 | 36% |
| Example 16 | 0.135 | 0.090 | 0.045 | 33% |
| Example 17 | 0.170 | 0.108 | 0.062 | 35% |
| Example 18 | 0.160 | 0.100 | 0.060 | 38% |
| Example 19 | 0.190 | 0.130 | 0.060 | 32% |
| Example 20 | 0.187 | 0.120 | 0.067 | 36% |
| Comparative | 0.180 | 0.135 | 0.045 | 25% |
| Example 3 | | | | |
| Comparative | 0.170 | 0.130 | 0.040 | 24% |
| Example 4 | | | | |

### Examples 21 and 22, and Comparative Examples 5 to 8

According to the exemplary embodiment, as shown in the conceptual diagram of FIG. 1(C), use is made of an active hydrogen generator in the form in which magnesium metal 4-1, the hydrogen molecule dissociative adsorption catalyst 4-2 of Table 7, and the salt of Table 8 were added to high concentration hydrogen-dissolved water 3 obtained by bubbling hydrogen gas into ion-exchanged water, which is placed in a drinking vessel 1 such as a PET bottle.

TABLE 7

| | Catalyst | Acid treatment |
|---|---|---|
| Example 21 | Platinum-supported ceramic | Untreated |
| Example 22 | Platinum-supported ceramic | Untreated |
| Comparative | No catalyst | Untreated |
| Example 5 | | |
| Comparative | No catalyst | Untreated |
| Example 6 | | |
| Comparative | No catalyst | Untreated |
| Example 7 | | |
| Comparative | No catalyst | Untreated |
| Example 8 | | |

TABLE 8

| | | Salt concentration |
|---|---|---|
| | Salt | (mmol/L) |
| Example 21 | CaSO₄ | 0.7 |
| Example 22 | MgSO₄ | 1.4 |
| Comparative | no Salt | |
| Example 5 | | |
| Comparative | CaSO₄ | 0.7 |
| Example 6 | | |
| Comparative | K₂SO₄ | 1.4 |
| Example 7 | | |
| Comparative | Na₂SO₄ | 1.4 |
| Example 8 | | |

The platinum-supported ceramic used in the hydrogen molecule dissociative adsorption catalyst of the active hydrogen generators of Examples 21 and 22 is the same as the hydrogen molecule dissociative adsorption catalyst of Example 2.

In a PET bottle containing a high concentration hydrogen-dissolved water which was obtained by bubbling hydrogen into 2000 mL of ion-exchanged water, 7 g of magnesium metal, 7 g of each of the hydrogen molecule dissociative adsorption catalysts of Examples 21 and 22 and Comparative Examples 5 to 8, and the salt were added, and the PET bottle was left to stand for 48 hours at room temperature. Subsequently, the magnesium metal and the hydrogen molecule dissociative adsorption catalyst were removed, the amounts of dissolved hydrogen were analyzed in the same manner as in Example 1, and thus Ax, Bx and Cx were determined. A summary of the values of Ax, Bx and Cx is presented in Table 9.

TABLE 9

| | Ax | Bx | Cx | Cx/Ax [%] |
|---|---|---|---|---|
| Example 21 | 0.720 | 0.470 | 0.250 | 35% |
| Example 22 | 0.780 | 0.510 | 0.270 | 35% |
| Comparative | 0.780 | 0.780 | 0.000 | 0% |
| Example 5 | | | | |
| Comparative | 0.720 | 0.720 | 0.000 | 0% |
| Example 6 | | | | |
| Comparative | 0.800 | 0.800 | 0.000 | 0% |
| Example 7 | | | | |
| Comparative | 0.720 | 0.720 | 0.000 | 0% |
| Example 8 | | | | |

### Examples 23 to 25

According to the present exemplary embodiment, as shown in the conceptual diagram of FIG. 1(A), use is made of an active hydrogen generator in the form in which tap water 3, magnesium metal 4-1, and the hydrogen molecule dissociative adsorption catalyst 4-2 of Table 10 are added to a drinking vessel 1 such as a PET bottle.

TABLE 10

| | Catalyst | Acid treatment |
|---|---|---|
| Example 23 | ZrO₂TiO₂-containing ceramic | Treated |
| Example 24 | ZrO₂-containing ceramic | Treated |
| Example 25 | Platinum-supported ceramic | Untreated |

The ZrO₂TiO₂-containing ceramic used in the hydrogen molecule dissociative adsorption catalyst of Example 23 is the same as the hydrogen molecule dissociative adsorption catalyst of Example 5.

The ZrO₂-containing ceramic used in the hydrogen molecule dissociative adsorption catalyst of Example 24 is the same as the hydrogen molecule dissociative adsorption catalyst of Example 1.

The platinum-supported ceramic used in the hydrogen molecule dissociative adsorption catalyst of Example 25 is the same as the hydrogen molecule dissociative adsorption catalyst of Example 2.

In a PET bottle containing 2000 mL of tap water, 7 g of magnesium metal and 7 g of each of the hydrogen molecule dissociative adsorption catalysts of Examples 23 to 25 were added, and the PET bottle was left to stand for 48 hours at room temperature. Subsequently, the magnesium metal and the hydrogen molecule dissociative adsorption catalyst were removed, the amounts of dissolved hydrogen were analyzed in the same manner as in Example 1, and thus Ax, Bx and Cx were determined. A summary of the values of Ax, Bx and Cx is presented in Table 11.

TABLE 11

| | Ax | Bx | Cx | Cx/Ax [%] |
|---|---|---|---|---|
| Example 23 | 0.780 | 0.440 | 0.340 | 44% |
| Example 24 | 0.780 | 0.510 | 0.270 | 35% |
| Example 25 | 0.780 | 0.510 | 0.270 | 35% |

### Comparative Example 9

According to the present exemplary embodiment, the concentration of dissolved hydrogen of hydrogen-dissolved water obtained by dissolving a high concentration of hydrogen in a commercially available aluminum pouch vessel was measured.

The concentration of dissolved hydrogen was measured since immediately after the opening of the vessel, and unlike the active hydrogen-dissolved water of the present invention, an inflection point representing a change in the concentration of dissolved hydrogen appeared after 10 hours. In this regard, it is speculated that since the water temperature for measurement was about 26° C., the release time was shortened. In addition, since the water of Comparative Example 9 did not contain a catalyst generating active hydrogen, or the like, except for the minerals included as impurities, it is believed that the amount of dissolved active hydrogen is very small.

### Reference Examples 1 to 4, and Comparative Examples 10 and 11

According to the present exemplary embodiment, the salt of Table 12 was added to ion-exchanged water, and constant current electrolysis was carried out at the current densities indicated in Table 12. Thus, the concentration of dissolved hydrogen in the water after electrolysis was measured.

TABLE 12

| | | Salt | |
|---|---|---|---|
| | | concentration | Current density |
| | Salt | (mmol/L) | (mA/cm²) |
| Reference | CaSO₄ | 0.7 | 3.5 |
| Example 1 | | | |
| Reference | CaSO₄ | 2.1 | 3.5 |
| Example 2 | | | |
| Reference | MgSO₄ | 1.3 | 6.5 |
| Example 3 | | | |
| Reference | MgSO₄ | 4.0 | 6.5 |
| Example 4 | | | |
| Comparative | K₂SO₄ | 1.4 | 3.5 |
| Example 10 | | | |
| Comparative | Na₂SO₄ | 1.4 | 3.5 |
| Example 11 | | | |

After the electrolysis, the amounts of dissolved hydrogen were analyzed in the same manner as in Example 1, and thus Ax, Bx and Cx were determined. A summary of the values of Ax, Bx and Cx is presented in Table 13.

TABLE 13

| | Ax | Bx | Cx | Cx/Ax [%] |
|---|---|---|---|---|
| Reference | 0.350 | 0.280 | 0.070 | 20% |
| Example 1 | | | | |
| Reference | 0.350 | 0.230 | 0.120 | 34% |
| Example 2 | | | | |
| Reference | 0.400 | 0.340 | 0.060 | 15% |
| Example 3 | | | | |
| Reference | 0.400 | 0.260 | 0.140 | 35% |
| Example 4 | | | | |
| Comparative | 0.380 | 0.380 | 0.000 | 0% |
| Example 10 | | | | |
| Comparative | 0.350 | 0.350 | 0.000 | 0% |
| Example 11 | | | | |

### Examples 26 to 27, and Comparative Example 12

According to the present exemplary embodiment, as shown in the conceptual diagram of FIG. 1(D), use is made of an active hydrogen generator in the form in which the electrolyzed water 3 obtained in Reference Example 1 and the hydrogen molecule dissociative adsorption catalyst 4 of Table 14 are added to a drinking vessel 1 such as a PET bottle.

TABLE 14

| | Catalyst | Acid treatment |
|---|---|---|
| Example 26 | Platinum-supported ceramic | Untreated |
| Example 27 | ZrO₂TiO₂-containing ceramic | Untreated |
| Comparative | No catalyst | Untreated |
| Example 12 | | |

The platinum-supported ceramic used in the hydrogen molecule dissociative adsorption catalyst of Example 26 is the same as the hydrogen molecule dissociative adsorption catalyst of Example 2.

The ZrO₂TiO₂-containing ceramic used in the hydrogen molecule dissociative adsorption catalyst of Example 27 is the same as the hydrogen molecule dissociative adsorption catalyst of Example 5.

In a PET bottle containing 2000 mL of the electrolyzed water obtained in Reference Example 1, 7 g of the hydrogen molecule dissociative adsorption catalyst was introduced, and the water was stirred for 5 minutes at room temperature. Subsequently, the hydrogen molecule dissociative adsorption catalyst was removed, the amounts of dissolved hydrogen were analyzed in the same manner as in Example 1, and thus Ax, Bx and Cx were determined. A summary of the values of Ax, Bx and Cx is presented in Table 15.

| | Ax | Bx | Cx | Cx/Ax [%] |
|---|---|---|---|---|
| Example 26 | 0.350 | 0.230 | 0.120 | 34% |
| Example 27 | 0.350 | 0.210 | 0.140 | 40% |
| Comparative | 0.350 | 0.280 | 0.070 | 20% |
| Example 12 | | | | |

The active hydrogen generators used in the Examples of the present invention all continuously generated active hydrogen at a high concentration, without requiring any maintenance such as washing with edible vinegar, for several months.

Furthermore, for all of the Examples and Comparative Examples, the active hydrogen-dissolved water obtained by inserting each of the active hydrogen generators and allowing the water to stand for 48 hours, was subjected to an ammonia test using Nessler's reagent, and it was confirmed that the solution was lightly colored red brown. Here, since ammonia is produced by a method such as the Haber-Bosch method, and thus, it is not apt to think that under the conditions of normal temperature and normal pressure, hydrogen molecules react with nitrogen molecules, thereby producing ammonia. Furthermore, although an ammonia test is carried out in the same manner as in the case of tap water, the presence of ammonia is not confirmed. Therefore, it is speculated that the active hydrogen reacted with nitrogen that was dissolved in water in a small amount, and thus ammonia was produced.

For all of the active hydrogen-dissolved water of Examples and Comparative Examples, the Nessler's reagent underwent less discoloration, and the taste or odor was satisfactory.

The embodiments of the production of active hydrogen-dissolved water as described above are only exemplary, and any forms of the method for producing active hydrogen and the active hydrogen generator, which have the constitution of the present invention and can be appropriately modified in design by a person having ordinary skill in the art, are all included in the scope of the present invention.

### EXPLANATIONS OF LETTERS OR NUMERALS

1 DRINKING WATER VESSEL
2 ACTIVE HYDROGEN GENERATING VESSEL
2-1 MAGNESIUM METAL
2-2 HYDROGEN MOLECULE DISSOCIATIVE ADSORPTION CATALYST
3 WATER
4 HYDROGEN MOLECULE DISSOCIATIVE ADSORPTION CATALYST
4-1 MAGNESIUM METAL
4-2 HYDROGEN MOLECULE DISSOCIATIVE ADSORPTION CATALYST
5 ACTIVE HYDROGEN GENERATING VESSEL
6 AGGREGATES OF MAGNESIUM METAL AND CALCIUM SULFATE
7 HYDROGEN MOLECULE DISSOCIATIVE ADSORPTION CATALYST
8 OPENING
9 OPENING
10 STOPPER
11 HYDROGEN MOLECULE DISSOCIATIVE ADSORPTION CATALYST VESSEL
12 AQUEDUCT
13 HYDROGEN MOLECULE DISSOCIATIVE ADSORPTION CATALYST
14 FILTER
15 HYDROGEN MOLECULE DISSOCIATIVE ADSORPTION CATALYST LAYER
16 HYDROGEN MOLECULE DISSOCIATIVE ADSORPTION CATALYST
17 OTHER FILTERED LAYER

1. A method for producing active hydrogen-dissolved water, comprising contacting a hydrogen molecule dissociative adsorption catalyst with an aqueous solution;
wherein:
the aqueous solution comprises water and at least one of calcium ion and magnesium ion;
the water is either:
(1) in contact with magnesium metal;
(2) dissolving hydrogen gas, by bubbling or applying high pressure; or
(3) being electrolyzed; and
the hydrogen molecule dissociative adsorption catalyst either:
(a) decomposes hydrogen into an active hydrogen in the aqueous solution, and is held within catalyst holding vessel, or
(b) decomposes hydrogen into an active hydrogen in the aqueous solution, and retains the water for a certain time period.

2. The method of claim 1, wherein the water of the items (2) and (3) is water that has been in contact with magnesium metal.

3. The method of claim 1, wherein the water is further brought into contact with magnesium metal inside the catalyst holding vessel.

4. The method of claim 1, wherein the hydrogen molecule dissociative adsorption catalyst comprises at least one catalyst selected from the group consisting of palladium, platinum, rhodium, ruthenium, zinc, zirconium, titanium, hafnium, vanadium, niobium, tungsten, iron, ruthenium oxide, rhodium oxide, copper oxide, zinc oxide, zirconium oxide, silicon dioxide, titanium oxide, hafnium oxide, aluminum oxide, vanadium oxide, niobium oxide, tungsten oxide, and iron oxide.

5. The method of claim 4, wherein the hydrogen molecule dissociative adsorption catalyst comprises at least one metal oxide selected from the group consisting of silicon dioxide, aluminum oxide, zirconium oxide, and titanium dioxide.

6. The method of claim 5, wherein the hydrogen molecule dissociative adsorption catalyst has been treated with an acid in advance.

7. The method of claim 6, wherein the acid treatment is conducted with an acid at a pH in the range from 2.5 or more to 4.5 or less.

8. An active hydrogen generator, comprising:
a hydrogen molecule dissociative adsorption catalyst decomposing hydrogen into an active hydrogen; and
a catalyst holding vessel holding the hydrogen molecule dissociative adsorption catalyst.

9. The active hydrogen generator of claim 8, further comprising magnesium metal in the catalyst holding vessel.

10. An active hydrogen generator, comprising a hydrogen molecule dissociative adsorption catalyst decomposing hydrogen into an active hydrogen in an aqueous solution and retaining water for a certain time period.

11. The active hydrogen generator of claim 8, wherein the hydrogen molecule dissociative adsorption catalyst comprises at least one catalyst selected from the group consisting of palladium, platinum, rhodium, ruthenium, zinc, zirconium, titanium, hafnium, vanadium, niobium, tungsten, iron, ruthenium oxide, rhodium oxide, copper oxide, zinc oxide, zirconium oxide, silicon dioxide, titanium oxide, hafnium oxide, aluminum oxide, vanadium oxide, niobium oxide, tungsten oxide, and iron oxide.

12. The active hydrogen generator of claim 8, further comprising at least one calcium compound selected from the group consisting of calcium sulfate anhydride, calcium sulfate hemihydrate, and calcium sulfate dehydrate generator.

13. The active hydrogen generator of claim 8, wherein the hydrogen molecule dissociative adsorption catalyst comprising at least one solid acid.

14. The active hydrogen generator of claim 13, wherein the solid acid is at least one acid selected from the group consisting of silicon dioxide, aluminum oxide, zirconium oxide, and titanium dioxide.

15. The active hydrogen generator of claim 14, wherein the hydrogen molecule dissociative adsorption catalyst has been treated with an acid in advance.

16. The active hydrogen generator according to claim 15, wherein the acid treatment is conducted with an acid at a pH in the range from 2.5 or more to 4.5 or less.

17. The active hydrogen generator of claim 10, wherein the hydrogen molecule dissociative adsorption catalyst comprises at least one catalyst selected from the group consisting of palladium, platinum, rhodium, ruthenium, zinc, zirconium, titanium, hafnium, vanadium, niobium, tungsten, iron, ruthenium oxide, rhodium oxide, copper oxide, zinc oxide, zirconium oxide, silicon dioxide, titanium oxide, hafnium oxide, aluminum oxide, vanadium oxide, niobium oxide, tungsten oxide, and iron oxide.

18. The active hydrogen generator of claim 10, further comprising at least one calcium compound selected from the group consisting of calcium sulfate anhydride, calcium sulfate hemihydrate, and calcium sulfate dehydrate.

19. The active hydrogen generator of claim 10, wherein the hydrogen molecule dissociative adsorption catalyst comprises at least one solid acid.

20. The active hydrogen generator of claim 19, wherein the solid acid is at least one acid selected from the group consisting of silicon dioxide, aluminum oxide, zirconium oxide, and titanium dioxide.

21. The active hydrogen generator of claim 20, wherein the hydrogen molecule dissociative adsorption catalyst has been treated with an acid in advance.

22. The active hydrogen generator of claim 21, wherein the acid treatment is conducted with an acid at a pH in the range from 2.5 or more to 4.5 or less.
